(19) United States
(12) Patent Application Publication
(10) Pub. No.: US 2008/0194779 A1
(43) Pub. Date: Aug. 14, 2008

(54) Process for the Manufacture of Chlorodifluoromethane

(75) Inventors: Mark John Christmas, Goshen, KY (US); Yiannis Nicolas Dimitratos, Wilmington, DE (US)

Correspondence Address: Hieser, David E.;E.I. Du Pont De Nemours and Company, Legal Patent Records Center, 4417 Lancaster Pike, Wilmington, DE, 19805, US

(73) Assignee: E.I. DU PONT DE NEMOURS AND COMPANY, WILMINGTON DELAWARE, DE (US)

(21) Appl. No.: 11/658,207

(22) PCT Filed: Oct. 15, 2004
(86) PCT No.: PCT/US04/34343

§ 371 Date: Jan. 23, 2007

Related U.S. Application Data
(60) Provisional application No. 60/602,429, filed on Aug. 18, 2004.

(51) Int. Cl. C07C 21/185; C07C 17/20; C07C 17/26; C07C 19/08; C08F 214/18

(52) U.S. Cl. 526/75; 570/163; 570/136; 570/153; 570/134; 252/182.15; 526/254

(57) ABSTRACT

A process is disclosed for the manufacture of CHClF₂ which involves contacting CHCl₃, HF and pentavalent antimony catalyst in the liquid phase; passing reactor vapor effluent to a reflux column to produce a reflux column vapor effluent of CHClF2 and HCl; passing the reflux column vapor effluent to a condenser to produce a condenser liquid effluent of CHClF₂ and a condenser vapor effluent of CHClF₂ and HCl; passing the condenser liquid effluent to the reflux column upper end; and recovering CHClF₂ from the condenser vapor effluent. The concentration of CHCl₂F and CHF₃ in the condenser vapor effluent is controlled by: (i) controlling the temperature at a point within the lower third of the theoretical stages of the reflux column by controlling the heat input to the reactor liquid phase; (ii) controlling the pressure in the reactor, reflux column and condenser by controlling the rate at which the condenser vapor effluent is removed from the condenser; (iii) maintaining the reflux ratio of the condenser at a substantially constant value; and (iv) maintaining the reactor liquid phase at substantially the maximum mass that does not result in entrainment or flooding of the reflux column. Also disclosed is CHClF₂ which is a product of this process. Also disclosed is a refrigerant comprising CHClF₂ and a method for its manufacture, a polymer foam blowing blend comprising CHClF₂ and a method for its manufacture, fluoromonomers tetrafluoroethylene and hexafluoropropylene produced by using CHClF₂ and a method for their manufacture, and a fluoropolymer produced by using CHClF₂ as a fluoromonomer precursor and a method for its manufacture; all involving the manufacture of CHClF₂ in accordance with the above process.

### FIELD OF THE INVENTION

The present invention relates generally to a process for the manufacture of chlorodifluoromethane, and more specifically to a process for the manufacture of chlorodifluoromethane wherein the concentration of undesirable dichlorofluoromethane and trifluoromethane in the chlorodifluoromethane product is controlled by using certain process parameters.

### BACKGROUND

Trifluoromethane (HFC-23, CHF₃) is generated as an undesirable by-product during the manufacture of chlorodifluoromethane (HCFC-22, CHClF₂). CHF₃ has a global warming potential of 11,700 over a 100-year time horizon, so its potential impact on climate change is significant. CHF₃ is said to be the second largest contributor to greenhouse gas emissions in the United States within the category of hydrofluorocarbons (HFCs), perfluorocarbons (PFCs), and SF₆.

CHClF₂ is produced in several developed and developing countries and is used as a refrigerant, a blend component in polymer foam blowing and as a precursor for the manufacture of fluoropolymers. CHClF₂ is produced by the reaction of chloroform (CHCl₃) and hydrogen fluoride (HF) in the presence of pentavalent antimony catalyst. Antimony pentachloride (SbCl₅) is the common catalyst precursor and pentavalent antimony species derived therefrom achieve a steady-state concentration in the reaction mixture that depend on residence time, temperature, and concentration of materials in the reactor. The reaction of SbCl₅ catalyst precursor and HF produces antimony chlorofluorides, SbCl₅₋ₓFₓ (where x is from 1 to 5), which react with the chlorinated compounds resulting in replacement of chlorine atoms with fluorine.

The stoichiometric relationship for the production of CHClF₂ from CHCl₃ and HF is:

CHCl₃+2 HF→CHClF₂+2 HCl

The reaction is commonly carried out in a continuous-flow reactor at elevated pressure and temperature. The HF and CHCl₃ are introduced into the reactor, which contains the catalyst in a liquid phase mixture of CHCl₃ and partially fluorinated intermediates. Although the reaction is exothermic, heat is added to increase the flow of vapors containing the CHClF₂ product from the reactor. The vapor stream leaving the reactor contains CHClF₂, dichlorofluoromethane (HCFC-21, CHCl₂F), CHF₃, HCl, CHCl₃, HF and some entrained antimony catalyst. Subsequent processing of this vapor commonly involves various separation processes to remove/recover by-products and to purify the CHClF₂ product. Generally, unreacted CHCl₃ and under-fluorinated intermediate CHCl₂F from the vapor stream are condensed in a condenser affixed to a reflux column and returned directly to the reactor, where they undergo fluorination to obtain additional CHClF₂ product. Any entrained antimony catalyst is also returned to the reactor. As shown in Table 1, the separation of the halomethanes is facilitated by their differences in volatility. As CHCl₃ is fluorinated, the addition of each fluorine atom results in a significant decrease in the normal boiling point of the resulting product.

TABLE 1
Major Components of Vapor from CHClF₂ Reactor

| Major Components | Normal Boiling | |
|---|---|---|
| Leaving the CHClF₂ | Point | |
| Reactor | (° C.) | Fate of Component |
| CHCl₃ | 61 | Recycled to reactor |
| HF | 19 | Recycled to reactor |
| CHCl₂F | 9 | Recycled to reactor |
| CHClF₂ | −41 | Recovered as product |
| CHF₃ | −82 | Separated from |
| | | CHClF₂ as a vapor and |
| | | is considered waste, or |
| | | is captured for use |
| HCl | −85 | Recovered as a by- |
| | | product |

Vapors leaving the condenser comprise major amounts of CHClF₂ and HCl, as well as residual HF and minor amounts of CHF₃ and CHCl₂F. In subsequent processing steps, HCl is recovered as a useful by-product and the HF can be removed by various methods. The CHClF₂ product is purified, typically by further distillation, caustic and water washing to remove residual acids, and drying to remove traces of water. By-product CHF₃ is separated as a vapor from the CHClF₂ and is commonly waste; but it can be captured for use in a limited number of applications (e.g., refrigeration or fire extinguishing). CHCl₂F leaving the condenser is problematic in that it is similar in volatility to CHClF₂ and will remain with the CHClF₂ product throughout the majority of the separation process. CHCl₂F is commonly separated from the CHClF₂ product in a downstream drying column. CHCl₂F accumulates in the bottom of the drying column and must be purged as a bottoms cut from the drying column to avoid contaminating the CHClF₂ product exiting the drying column as an overhead stream. A significant amount of CHClF₂ can be lost with the CHCl₂F so purged, which negatively impacts process yields of CHClF₂.

The quantity of CHF₃ produced during the production of CHClF₂ depends on how the process is operated. For example, research in the United States showed that at plants not fully optimized to reduce CHF₃ generation, the upper bound for CHF₃ emissions can be on the order of 3 to 4 percent of the CHClF₂ production.

There is an industry need for CHClF₂ manufacturing processes that advantageously allow control of both CHCl₂F and CHF₃ as CHClF₂ is produced, thereby allowing more efficient production of CHClF₂.

### SUMMARY OF THE INVENTION

A process for the manufacture of CHClF₂ is provided. The process comprises (a) contacting CHCl₃, HF and catalyst comprising pentavalent antimony in the liquid phase in a reactor to form a reactor liquid phase and a reactor vapor effluent comprising CHCl₃, CHCl₂F, HF, CHClF₂, CHF₃ and HCl; (b) passing the reactor vapor effluent to a reflux column having a lower and upper end, the reflux column lower end in fluid communication with the reactor, to produce a reflux column vapor effluent comprising CHClF₂ and HCl; (c) passing the reflux column vapor effluent from the reflux column upper end to a condenser in fluid communication with the reflux column upper end to produce a condenser liquid effluent comprising CHClF₂ and a condenser vapor effluent comprising CHClF₂ and HCl; (d) controlling the concentration of CHCl₂F and CHF₃ in the condenser vapor effluent; (e) passing the condenser liquid effluent to the reflux column upper end; and (f) recovering CHClF₂ from the condenser vapor effluent. In accordance with this invention, in (d), the concentration of CHCl₂F and CHF₃ in the condenser vapor effluent can be controlled by (i) controlling the temperature at a point within the lower third of the theoretical stages of the reflux column by controlling the heat input to the reactor liquid phase; (ii) controlling the pressure in the reactor, the reflux column and the condenser by controlling the rate at which the condenser vapor effluent is removed from the condenser; (iii) maintaining the reflux ratio of the condenser at a substantially constant value; and (iv) maintaining the reactor liquid phase at substantially the maximum mass that does not result in entrainment or flooding of the reflux column. This invention also provides CHClF₂ which is a product of this process.

This invention further provides a refrigerant comprising CHClF₂ and a method for its manufacture, a polymer foam blowing blend comprising CHClF₂ and a method for its manufacture, fluoromonomers tetrafluroethylene and hexafluoropropylene produced by using CHClF₂ and a method for their manufacture, and a fluoropolymer produced by using CHClF₂ as a fluoromonomer precursor and a method for its manufacture; all involving the manufacture of CHClF₂ in accordance with the above process.

### BRIEF DESCRIPTION OF THE DRAWINGS

FIG. 1 is an illustrative plot (at five pressures) of the temperature at a point within the lower third of the theoretical stages of a reflux column versus the weight percent of CHCl₂F (HCFC-21) in condenser vapor effluent (based on the combined weight of CHCl₂F and CHClF₂ in the condenser vapor effluent) that might be obtained by operating one embodiment of a process in accordance with this invention.

FIG. 2 is an illustrative plot (at five pressures) of the temperature at a point within the lower third of the theoretical stages of a reflux column versus the weight percent of CHF₃ (HFC-23) in condenser vapor effluent (based on the combined weight of CHF₃ and CHClF₂ in the condenser vapor effluent) that might be obtained by operating the same embodiment of a process in accordance with this invention as in FIG. 1.

FIG. 3 is a schematic drawing representing a configuration of reactor, reflux column, and condenser system that can be used for practicing the process of the present invention.

FIG. 4 is an illustrative run plot of the weight percent of CHCl₂F (HCFC-21) in condenser vapor effluent (based on the combined weight of CHCl₂F and CHClF₂ in the condenser vapor effluent) versus time that might be obtained for two embodiments of processes in accordance with this invention.

FIG. 5 is an illustrative run plot of the weight percent of CHF₃ (HFC-23) in condenser vapor effluent (based on the combined weight of CHF₃ and CHClF₂ in the condenser vapor effluent) versus time that might be obtained for two embodiments of processes in accordance with this invention.

FIG. 6 is an illustrative run plot of the condenser vapor effluent CHF₃ ratio versus time that might be obtained for two embodiments of processes in accordance with this invention (B and C) and a comparative process (A).

### DETAILED DESCRIPTION

This invention provides a CHClF₂ manufacturing process wherein both CHCl₂F and CHF₃ can be readily controlled, thus allowing efficient production of CHClF₂. In particular, the process control used in this invention permits limiting the amount of by-product CHF₃ produced during the manufacture of CHClF₂ from CHCl₃ while at the same time limiting the amount of intermediate CHCl₂F exiting with the reflux column vapor effluent. The process control used in the present invention also facilitates stabilization of the reflux column composition profile and reflux column vapor effluent composition; and facilitates response to catalyst aging.

The process of the present invention involves contacting CHCl₃, HF and catalyst comprising pentavalent antimony in the liquid phase in a reactor to form a reactor liquid phase and a reactor vapor effluent comprising CHCl₃, CHCl₂F, HF, CHClF₂, CHF₃ and HCl.

The pentavalent antimony used as a catalyst in the present invention can be represented by the formula SbCl₅₋ₓFₓ, (wherein x is 1 to 5) and can be formed in a conventional fashion. For example, in the presence of HF under the conditions within the reactor, SbCl₅ can be used to form SbCl₅₋ₓFₓ, wherein x is 1 to 5. The amount of pentavalent antimony catalyst present in the reactor liquid phase is preferably from about 25 weight percent to about 65 weight percent, more preferably from about 25 weight percent to about 45 weight percent, of the reactor liquid phase. It has been found that for the process of this invention, lower pentavalent antimony catalyst concentrations within these ranges will typically produce less CHF₃ than higher catalyst concentrations, without substantial reduction in the CHClF₂ production rate. Accordingly, the amount of pentavalent antimony catalyst present in the reactor liquid phase is preferably as low as possible within these ranges, considering the size of the reactor and the rate of CHClF₂ production desired. However, operating at a high CHClF₂ production rate (i.e., by a high HF feed rate) with insufficient pentavalent antimony catalyst in the reactor is undesirable, as this can result in severe corrosion conditions in the reactor. Of note are embodiments wherein the amount of pentavalent antimony catalyst present in the reactor liquid phase is from about 25 weight percent to about 35 weight percent (e.g., from about 25 weight percent to about 30 weight percent) of the reactor liquid phase.

The minimum amount of pentavalent antimony catalyst necessary in the reactor liquid phase during the contacting of CHCl₃, HF and catalyst is typically determined based on the maximum intended HF feed rate. The minimum weight of pentavalent antimony catalyst in kg present in the reactor liquid phase is normally at least about two times the maximum intended hourly weight flow rate in kg/h of HF to the reactor liquid phase. The absolute amount of catalyst in the reactor liquid phase should be established based on the CHClF₂ production demand, and the reactor size will establish the lower pentavalent antimony catalyst concentration limit.

The age of pentavalent antimony catalyst in the reactor liquid phase negatively influences the production of CHF₃. The amount of CHF₃ produced will increase as the pentavalent antimony catalyst ages unless conditions are adjusted to compensate. At the point where CHClF₂ production rate becomes unreasonably slow, it is economically beneficial to replace the pentavalent antimony catalyst. Chlorine can also be fed to the reactor to oxidize any unreactive trivalent antimony catalyst back to the active pentavalent form.

The rate at which HF is added to the reactor during said contacting is normally less than about 0.5 kg of HF per hour, per kg of catalyst. The HF feed rate to the reactor can be used to set the CHClF₂ production rate for the process. Of note are embodiments of this invention where the CHClF₂ production rate is controlled by controlling the HF feed flowrate to the reactor. The weight ratio of CHCl₃ to HF added to the reactor during said contacting is normally from about 2.4 to about 3.2.

The stepwise reaction of CHCl₃ with HF in the presence of pentavalent antimony catalyst to form CHClF₂ is net exothermic. However, additional heat must ordinarily be supplied to the reactor liquid phase to obtain a commercially viable reaction rate as well as to generate sufficient reactor vapor effluent reaching the reflux column to facilitate the separation of lower boiling compounds (i.e., HCl (nBP −85° C.), CHF₃ (nBP -82° C.), and CHClF₂ (nBP −41° C.)) from higher boiling compounds (i.e., CHCl₂F (nBP 9° C.), HF (nBP 20° C.), and CHCl₃ (nBP 60° C.)) present in the reactor vapor effluent.

For safe and continuous operation of the present process it is desirable that the temperature of the reactor liquid phase be maintained above about 68° C. Under certain conditions, pentavalent antimony catalyst of the present invention and HF can undesirably form a superacid, a severely corrosive composition that causes reactors made of conventional materials of construction for HF service to erode and eventually fail. Such failure could ultimately result in a breach of the reactor and loss of containment leading to a release of hazardous materials. Such detrimental conditions in the reactor are avoided when the temperature of the reactor liquid phase is maintained at about 68° C. or more. Accordingly, the heat supplied to the reactor liquid phase is preferably adjusted to maintain the reactor liquid phase temperature at from about 68° C. to about 95° C. Heat can be supplied to the reactor liquid phase by conventional means.

The production of CHF₃ can be significantly affected by reactor liquid phase temperature. It has been found that for the process of this invention, an increase in the reactor liquid phase temperature can result in a decrease in the rate of CHF₃ production. Accordingly, a temperature of about 70° C. or more is the optimum temperature for minimum production of CHF₃, and a particularly preferred temperature range is from about 70° C. to about 90° C.

In accordance with this invention, the temperature at a point within the lower third of the theoretical stages of the reflux column is controlled by controlling the heat input to the reactor liquid phase. As noted above, heat can be supplied to the reactor liquid phase by conventional means. Examples include use of a heating jacket surrounding the reactor or use of a heating coil submerged in the reactor liquid phase. It has been found that for the process of this invention, the result of an increase in heat to the reactor liquid phase is observed more quickly in temperature change at a point within the lower third of the theoretical stages in the reflux column than either in temperature change of the reactor liquid phase or in temperature change at a point in the upper two thirds of the theoretical stages of the reflux column. Thus, temperature control of the present process from a point within the lower third of the theoretical stages in the reflux column is more responsive than temperature control based on temperature measurement at any other point in the reaction apparatus. For instance, such control avoids control of temperature in the upper section of the reflux column, which is problematic.

The temperature at a point within the lower third of the theoretical stages in the reflux column is typically controlled to be from about 30° C. to about 60° C. by controlling the heat input to the reactor liquid phase. The temperature of the reactor liquid phase is thus indirectly controlled, and as a result can vary somewhat, typically within the aforementioned range of about 68° C. to about 95° C. Normally the reactor liquid phase temperature is allowed to so vary while the temperature at a point within the lower third of the theoretical stages in the reflux column is held relatively constant (within the range of from about 30° C. to about 60° C.). The temperature of the reactor liquid phase is preferably monitored, but is normally used only to the extent to assure that it is at a desirable temperature for safe and continuous operation (e.g., a temperature above about 68° C. to maintain the structural integrity of the reactor as discussed above).

In accordance with this invention the reactor liquid phase is maintained at substantially the maximum mass that does not result in entrainment or flooding of reactor liquid phase into the reflux column. Ordinarily this is accomplished by controlling the CHCl₃ feed flowrate to the reactor. The maximum mass of reactor liquid phase with which to operate the present process will vary with a given reactor's size and configuration of internal components, but is easily determined without undue experimentation by those of ordinary skill in this field. The present invention includes the finding that maintaining the reactor liquid phase at maximum mass can result in less CHF₃ production than when the reactor liquid phase mass is maintained significantly below the maximum mass. Further, maintaining the reactor liquid phase mass significantly below the maximum mass can result in insufficient CHCl₃ and CHCl₂F in the reactor liquid phase, which in turn can result in a destructively corrosive environment in the reactor, especially at higher HF feed rates. A reactor liquid phase mass higher than the maximum mass will bring the liquid level in the reactor too close to the top of the reactor, and can lead to entrainment of reactor liquid phase containing pentavalent antimony catalyst into the lower end of the reflux column and/or flooding of the lower section of the reflux column. The presence of pentavalent antimony catalyst into the lower end of the reflux column can create a destructively corrosive environment in the lower end of the reflux column as the HF concentration is relatively high at this location. In addition, such entrainment or flooding of the lower section of the reflux column can impair the column separation performance and adversely affect the amount of CHCl₂F and CHF₃ present in the condenser vapor effluent.

In accordance with this invention, the reactor vapor effluent is passed to a reflux column having a lower and upper end, the reflux column lower end in fluid communication with the reactor, to produce a reflux column vapor effluent comprising CHClF₂ and HCl. Reflux columns of various designs may be used, including for example, columns that are filled with packing (packed columns) and columns that have internal trays (trayed columns). Columns that have internal configurations that are a combination of one or more packed segments and one or more trayed segments may also be used. Of note are processes wherein a lower portion of the reflux column is packed and an upper portion of the reflux column is trayed. Of particular note are reflux columns wherein the bottom one-third of the length of the reflux column contains packing, and the upper two-thirds of the length of the reflux column contains trays.

In accordance with this invention, the reflux column vapor effluent from the reflux column upper end is passed to a condenser in fluid communication with the reflux column upper end to produce a condenser liquid effluent comprising CHClF₂ and a condenser vapor effluent comprising CHClF₂ and HCl. Condensers of various designs may be used. Suitable condensers for carrying out the process of this invention include liquid-cooled condensers with no condensate holdup.

The reflux ratio (as used herein) is defined as the mass flow rate of condenser vapor effluent being removed from the condenser, divided by the mass flow rate of condenser liquid effluent passing to the reflux column upper end. The reflux ratio for the process of this invention is normally within the range of from about 1.0 to about 2.5, and is preferably within the range of from about 1.2 to about 2.0.

In accordance with this invention the reflux ratio of the condenser is maintained at a substantially constant value. Ordinarily, this is accomplished by contolling the cooling rate of the condenser. Typically a cooling fluid is used to cool the condenser, and the cooling fluid flow through the condenser is adjusted to produce sufficient condensate, which when returned to the reflux column, provides the desired reflux flow. Normally, the cooling fluid flow to the condenser is maintained constant when production rate is constant, but the cooling fluid flow (and thus cooling) can be increased as the CHClF₂ production rate is increased to maintain adequate separation in the reflux column. Sufficient reflux flow down the reflux column causes CHCl₂F from the reactor vapor phase traversing the reflux column to be substantially removed and results in formation of a reflux column vapor effluent exiting the reflux column upper end that is substantially free of CHCl₂F. If the reflux flow down the reflux column is too low, then CHCl₂F from the reactor vapor phase is not separated as the reactor vapor phase traverses the reflux column and will result in CHCl₂F exiting the reflux column upper end with the reflux column vapor effluent. Conversely, reflux flow down the reflux column should not be set at too high a rate as this will unnecessarily increase the rate of cooling fluid flow through the condenser, and will also generally necessitate additional heat input to the reactor to maintain the desired temperature at a point within the lower third of the theoretical stages of the reflux column, thereby undesirably and unnecessarily increasing production costs. An excessive reflux flow rate will also return a significant amount of CHClF₂ back into the reflux column (and likely on into the reactor where further fluorination can result in additional formation of the undesired byproduct CHF₃). Thus, cooling fluid flow through the condenser is adjusted to produce the desired reflux flow to cool the reflux column and to control the reflux ratio of the condenser so that the condenser liquid effluent comprises CHClF₂, and the condenser vapor effluent comprises mainly CHClF₂ and HCl, with the condenser vapor effluent being substantially free of CHCl₂F. Typically, the condenser liquid effluent also comprises HCl.

Where the production rate of the reactor is changed, the cooling fluid flow through the condenser should be appropriately adjusted to maintain a constant reflux ratio. Means for automatic adjustment may be provided within the process system. The optimum reflux ratio with which to operate the present process will vary with a given reflux column size, geometry, and internal configuration, but is easily determined without undue experimentation by those of ordinary skill in this field.

The reactor, reflux column and condenser components, herein also collectively referred to as the reaction apparatus, are in fluid communication and thus the pressure within these components is substantially identical, except for the normal pressure gradient across such an apparatus. In accordance with this invention, the pressure in the reactor, the reflux column and the condenser are controlled by controlling the rate at which the condenser vapor effluent is removed from the condenser. The condenser vapor effluent removal rate most directly controls pressures of the apparatus near to the condenser (e.g., the pressure at the upper end of the reflux column). It has been found that for the process of this invention, because the apparatus units are in fluid communication, the pressure throughout the apparatus can be effectively controlled by controlling the condenser vapor effluent removal rate.

The pressure of the reaction apparatus, measured at the reflux column upper end, is normally from about 1,411 kPa (190 psig) to about 1,687 kPa (230 psig). Operating the process of this invention at pressures outside of this range is possible, as the reaction apparatus operating pressure can be dependant on restrictions imposed by downstream apparatus or condenser design. For example, circulating −15° C. coolant in the condenser will allow for a lower reaction apparatus operating pressure than circulating 20° C. coolant. It has been found that for the process of this invention, operating at lower reaction apparatus pressure is desirable, as using higher reactor apparatus pressure can result in an undesirable increase in the amount of CHF₃ produced. For practical commercial processes, there will exist a lower reactor apparatus pressure limit, below which operation is not reasonable. This lower pressure limit can be imposed by downstream processing apparatus in fluid communication with the present reactor apparatus. For example, a distillation column for separation of HCl from CHClF₂ in the condenser vapor effluent can be in fluid communication with the condenser. This lower pressure limit can also be imposed by the availability of cooling fluid for the condenser as well as by the preferred embodiment where maximum mass of reaction liquid phase is maintained in the reactor.

It will be evident to one of ordinary skill in the art that although the reflux column and the condenser have been discussed herein separately, they need not necessarily be separate units. For example, the reflux column and the condenser can be integrated into a single column having a staged section toward its lower end and a cooled section near its upper end.

The pressure of the reaction apparatus can be controlled by adjusting the flow of condenser vapor effluent leaving the condenser.

CHClF₂ is recovered from the condenser vapor effluent. This may be accomplished by known processes, such as conventional distillation to separate CHClF₂ from HCl.

In accordance with this invention, the concentration of CHCl₂F and CHF₃ in the condenser vapor effluent can be controlled as described herein by (i) controlling the temperature at a point within the lower third of the theoretical stages of the reflux column; (ii) controlling the pressure in the reactor, the reflux column and the condenser; (iii) maintaining the reflux ratio of the condenser at a substantially constant value; and (iv) maintaining the reactor liquid phase at substantially the maximum mass that does not result in entrainment or flooding of the reflux column. It has been found that for the process of this invention, this unique combination of operating controls can provide advantageous control of both of CHCl₂F and CHF₃. This unique combination of operating controls (i), (ii), (iii) and (iv) may not only be used in conjunction with a newly designed CHClF₂ manufacturing system, but it may also be used to upgrade existing CHClF₂ manufacturing systems.

Of note are embodiments of this invention where specification limits for the permissible concentrations of CHCl₂F and CHF₃ in condenser vapor effluent produced by operating the process, as dictated by the economics of the process, are used. The process of this invention can comprise, for example, measuring the concentration of CHCl₂F and of CHF₃ in the condenser vapor effluent, and controlling the concentrations of CHCl₂F and CHF₃ in condenser vapor effluent as necessary to provide a condenser vapor effluent wherein the concentration of each is below a designated specification limit set for it. This includes embodiments wherein the concentration of CHCl₂F in the condenser vapor effluent is greater than a set specification limit, and the concentration of CHCl₂F in the condenser vapor effluent is reduced by reducing the temperature at a point within the lower third of the theoretical stages of the reflux column by reducing the heat input to the reactor liquid phase. Also included are embodiments wherein the concentration of CHF₃ in the condenser vapor effluent is greater than a set specification limit, and the concentration of CHF₃ in the condenser vapor effluent is reduced by reducing the pressure in the reactor, the reflux column and the condenser by increasing the rate at which the condenser vapor effluent is removed from the condenser.

Typically, at least for new manufacturing systems or upgraded existing manufacturing systems of relatively efficient design which operate in accordance with this invention, specification limits of about 0.75 weight percent CHF₃ or less (based on the combined weight of CHF₃ and CHClF₂ in the condenser vapor effluent) and about 0.1 weight percent CHCl₂F or less (based on the combined weight of CHCl₂F and CHClF₂ in the condenser vapor effluent) can be readily achieved. For many uses, and for the purposes of the present specification, condenser vapor effluent meeting these specification limits can be considered to be substantially free of CHCl₂F and CHF₃. Preferred processes include those which are controlled to produce a condenser vapor effluent having about 0.65 weight percent CHF₃ or less (based on the combined weight of CHF₃ and CHClF₂ in the condenser vapor effluent). Also preferred are processes which are controlled to produce a condenser vapor effluent having about 0.05 weight percent CHCl₂F or less (based on the combined weight of CHCl₂F and CHClF₂ in the condenser vapor effluent); with processes controlled to produce a condenser vapor effluent having about 0.02 weight percent CHCl₂F or less (based on the combined weight of CHCl₂F and CHClF₂ in the condenser vapor effluent) being particularly preferred. It is nevertheless noted that the specification limits which can be met for the concentrations of CHCl₂F and CHF₃ can also depend on the design of the manufacturing system. For a relatively less efficient manufacturing system design, specification limits corresponding to higher amounts of CHCl₂F and CHF₃ may be appropriate; and additional purification of the condenser vapor effluent may also be appropriate for such systems. The concentration of CHCl₂F and CHF₃ in the condenser vapor effluent can be determined by common analytical techniques, for example, gas chromatography or infrared spectroscopy.

It has been found that for the processes of this invention, the relationships shown in Table 2 between certain process parameters and the concentrations of CHCl₂F and CHF₃ in the condenser vapor phase may be observed.

TABLE 2

| As the following process | The concentration of | The concentration |
|---|---|---|
| parameters are increased | CHCl₂F in the | of CHF₃ in the |
| (all other variables held | condenser vapor | condenser vapor |
| constant): | phase will: | phase will: |
| Reactor apparatus pressure | Decrease | Increase |
| Condenser reflux ratio | Decrease | Increase |
| Temperature at a point | Increase | Decrease |
| within the lower third of the | | |
| theoretical stages in the | | |
| reflux column: | | |
| Reactor mass: | Decrease | Decrease |

The first three of the process parameters listed in Table 2 are opposite in direction of effect on the concentration of the two compounds, so that what decreases CHCl₂F concentration will increase CHF₃ concentration. One process parameter, reactor mass, is similar in direction of effect so that the optimum reactor mass, as stated earlier herein, is maximum mass that does not result in entrainment or flooding of reactor liquid phase into the reflux column.

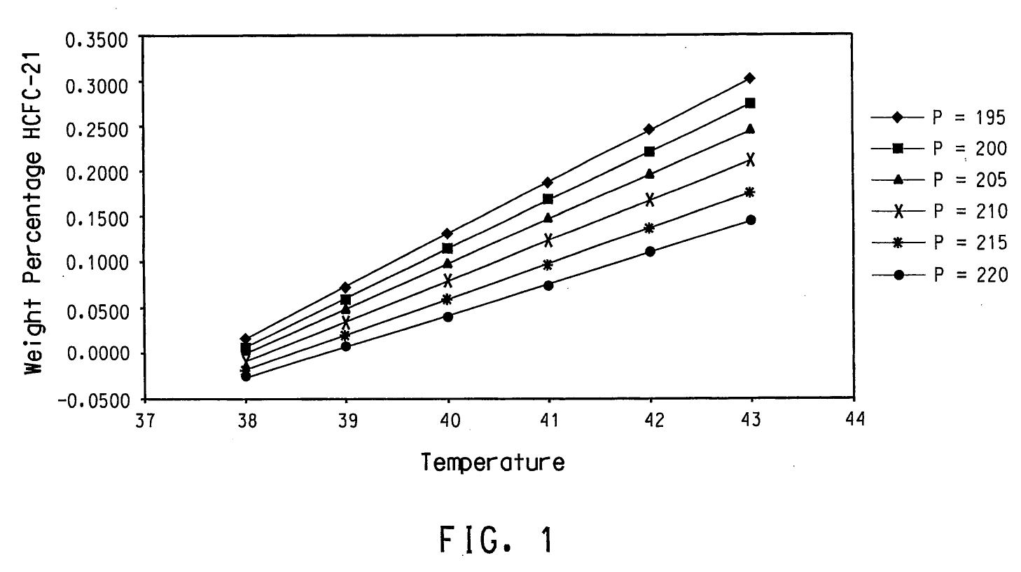

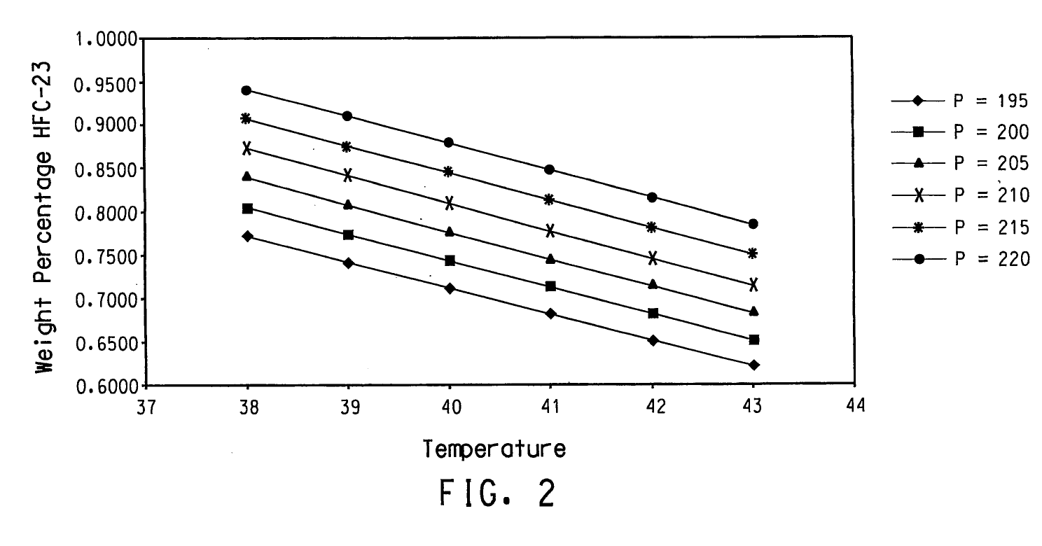

For the purpose of further illustrating the process of this invention FIG. 1 and FIG. 2 are provided to exemplify concentrations of CHCl₂F (FIG. 1) and CHF₃ (FIG. 2) in weight percent (based on the combined weight of each respectively with CHClF₂) at steady-state in an example condenser vapor effluent that might be obtained by operating a process of the present invention at a preferred constant reflux ratio (say, about 1.6). Reactor apparatus pressure (measured at the reflux column upper end) or temperature at a point within the lower third of the theoretical stages of the reflux column is varied while other variables are held constant, and the resultant concentrations of CHCl₂F and CHF₃ in the condenser vapor effluent are indicated. FIG.1 contains plots (at five pressures) of the concentration of CHCl₂F in condenser vapor effluent while the temperature at a point within the lower third of the theoretical stages of a reflux column is varied. FIG. 2 contains plots (at the same five pressures) of the corresponding concentration of CHF₃ in condenser vapor effluent while the temperature at a point within the lower third of the theoretical stages of a reflux column is varied.

It is evident from FIG. 1 that for the illustrated process the CHCl₂F concentration in the condenser vapor effluent is less sensitive to changes in reactor apparatus pressure at temperatures at the lower end of the range of temperatures at a point within the lower third of the theoretical stages in the reflux column. It is also evident from FIG. 1 and FIG. 2 together that for the illustrated process (1) a decrease in reactor apparatus pressure, especially at the lower end of the range of temperatures at a point within the lower third of the theoretical stages of a reflux column, correlates with a relatively large decrease in CHF₃ concentration in the condenser vapor effluent and a relatively small increase in CHCl₂F concentration in the condenser vapor effluent; and (2) a decrease in temperature at a point within the lower third of the theoretical stages of a reflux column correlates with a relatively large decrease in CHCl₂F concentration in the condenser vapor effluent and a relatively small increase in CHF₃ concentration in the condenser vapor effluent.

As noted above, reduced amounts of CHCl₂F and CHF₃ in the condenser vapor effluent are achieved in accordance with this invention by maintaining the reactor liquid phase at maximum mass that does not result in entrainment or flooding of the reflux column. Once the liquid phase mass level in the reactor has been set, further reduction of the concentration of CHCl₂F and/or CHF₃ in the condenser vapor effluent can be achieved by controlling the temperature at a point within the lower third of the theoretical stages in said reflux column by controlling the heat input to said reactor liquid phase; and controlling the pressure in the reaction apparatus by controlling the rate at which said condenser vapor effluent is removed from said condenser. In the event specification limits for CHCl₂F and/or CHF₃ are not being achieved during the manufacture of CHClF₂ in accordance with this invention, appropriate adjustment of the temperature at a point within the lower third of the theoretical stages in the reflux column and of the reactor apparatus pressure may readily be made. The amount of CHCl₂F or CHF₃ in the condenser vapor effluent can be further reduced as the reflux ratio of the condenser is set. However, the reflux ratio of the condenser is maintained substantially constant during steady state operation of the present process, and is itself a function of the of the rate at which the condenser vapor effluent is removed from the condenser.

The process of this invention provides a convenient means for adjusting a CHClF₂ manufacturing process such that the steady-state concentration of CHCl₂F and/or CHF₃ in the condenser vapor effluent is maintained below appropriate specification limits. By implementing the process of this invention, many CHClF₂ manufacturing systems can achieve a condenser vapor effluent comprising CHClF₂ and HCl which is substantially free of both CHCl₂F and CHF₃. Practice of the process of this invention is further illustrated by the two general scenarios which follow.

In the first general scenario, the concentration of CHCl₂F in the condenser vapor effluent is higher than the CHCl₂F specification and the concentration of CHF₃ is within the CHF₃ specification. The concentration of CHCl₂F can be reduced to within the CHCl₂F specification by reducing the temperature at a point within the lower third of the theoretical stages in the reflux column. This temperature is reduced by reducing the heat input to the reactor liquid phase. The temperature at a point within the lower third of the theoretical stages in the reflux column can be reduced, and thereby the CHCl₂F concentration, as long as the CHF₃ concentration remains within specification. If this temperature reduction causes the temperature of the reactor liquid phase to approach about 68° C., then such temperature reduction is halted and the reaction liquid phase temperature maintained at about 68° C. or above, and then the reaction apparatus pressure is increased by reducing the flow of condenser vapor effluent from the condenser until the concentration of CHCl₂F in the condenser vapor effluent is reduced to within the CHCl₂F specification.

In the second general scenario, the concentration of CHF₃ in the condenser vapor effluent is determined to be higher than the CHF₃ specification, and the concentration of CHCl₂F is determined to be lower than the CHCl₂F specification. The concentration of CHF₃ can be reduced to within the CHF₃ specification by decreasing the reaction apparatus pressure by increasing the rate of removal of condenser vapor effluent from the condenser. This pressure can be reduced, thereby reducing the CHF₃ concentration in the condenser vapor effluent, as long as the CHCl₂F concentration in the condenser vapor effluent does not rise and exceed CHCl₂F specification. If this pressure reduction causes the reaction apparatus pressure to reach the minimum pressure at which the reaction apparatus can be operated before the amount of CHF₃ in the condenser vapor effluent is within the CHF₃ specification, then the temperature at a point within the lower third of the theoretical stages in the reflux column is increased by increasing the heat input to the reactor liquid phase.

This invention includes CHClF₂ which is a product of the process of this invention. CHClF₂ can be used as a refrigerant; and this invention provides a refrigerant comprising CHClF₂ manufactured by the process of this invention. Typically, CHClF₂ is used as a refrigerant in combinations that also include at least one compound selected from the group consisting of carbon dioxide, ammonia, hydrocarbons, hydrofluorocarbons, hydrochlorofluorocarbons, perfluorocarbons, hydrofluoroethers and perfluoroethers. Accordingly, this invention includes a refrigerant comprising (a) CHClF₂ manufactured by the process of this invention; and (b) at least one compound selected from the group consisting of carbon dioxide, ammonia, hydrocarbons, hydrofluorocarbons, hydrochlorofluorocarbons, perfluorocarbons, hydrofluoroethers and perfluoroethers. This invention also provides a method of producing a refrigerant which comprises manufacturing CHClF₂ in accordance with the process described herein; and mixing CHClF₂ manufactured in accordance with the process described herein with at least one compound selected from the group consisting of carbon dioxide, ammonia, hydrocarbons, hydrofluorocarbons, hydrochlorofluorocarbons, perfluorocarbons, hydrofluoroethers and perfluoroethers. Of note are embodiments wherein the combinations comprise CHClF₂ and at least one compound selected hydrocarbons, hydrofluorocarbons, hydrochlorofluorocarbons and perfluorocarbons having from 2 to 4 carbon atoms.

CHClF₂ can be used as a component of a blend used for polymer foam blowing; and this invention provides a polymer foam blowing blend comprising CHClF₂ manufactured in accordance with the process of this invention. This invention includes a polymer foam blowing blend comprising (a) CHClF₂ manufactured by the process of this invention, and (b) at least one compound selected from the group consisting of dimethyl ether, carbon dioxide, hydrocarbons, hydrofluorocarbons, hydrochlorofluorocarbons, perfluorocarbons, hydrofluoroethers and perfluoroethers. This invention also provides a method of producing a polymer foam blowing blend which comprises manufacturing CHClF₂ in accordance with the process described herein; and blending CHClF₂ manufactured in accordance with the process described herein with at least one compound selected from the group consisting of dimethyl ether, carbon dioxide, hydrocarbons, hydrofluorocarbons, hydrochlorofluorocarbons, perfluorocarbons, hydrofluoroethers and perfluoroethers. Of note are embodiments wherein the blends comprise CHClF₂ and at least one compound selected hydrocarbons, hydrofluorocarbons, hydrochlorofluorocarbons and perfluorocarbons having from 1 to 4 carbon atoms.

CHClF₂ manufactured in accordance with the process of this invention can be used as a precursor for the manufacture of the fluoromonomer tetrafluoroethylene and the fluoromonomer hexafluoropropylene; and thus as a precursor to fluoropolymers produced using those at least one of those fluoromonomers. Tetrafluoroethylene and/or hexafluoropropylene may be produced from CHClF₂ by pyrolysis. Accordingly, this invention includes a compound of the formula CF₂═CFX, wherein X is selected from the group consisting of F and CF₃, produced by pyrolyzing CHClF₂ manufactured in accordance with the process of this invention. This invention further includes a method for producing at least one compound of the formula CF₂═CFX, wherein X is selected from the group consisting of F and CF₃, comprising manufacturing CHClF₂ in accordance with the process described herein; and pyrolyzing CHClF₂ manufactured in accordance with the process described herein. This invention also provides a method of producing a fluoropolymer which comprises (1) manufacturing CHClF₂ in accordance with the process described herein; (2) pyrolyzing CHClF₂ manufactured in accordance with the process described herein to produce at least one compound of the formula CF₂═CFX, wherein X is selected from the group consisting of F and CF₃; and (3) polymerizing at least one compound of the formula CF₂═CFX produced by pyrolyzing the CHClF₂ manufactured in accordance with the process described herein, optionally together with at least one comonomer. This invention also includes a fluoropolymer produced by that method.

### EXAMPLES

“CHF₃ ratio” is defined as the mass of CHF₃ in the condenser vapor effluent divided by the mass of CHClF₂ in the condenser vapor effluent, expressed as a percentage.

“CHF₃ concentration” is defined as the mass of CHF₃ divided by the combined mass of CHF₃ and CHClF₂ in the condenser vapor effluent, expressed as a percentage.

“CHCl₂F concentration” is defined as the mass of CHCl₂F divided by the combined mass of CHCl₂F and CHClF₂ in the condenser vapor effluent, expressed as a percentage.

### Reaction Apparatus

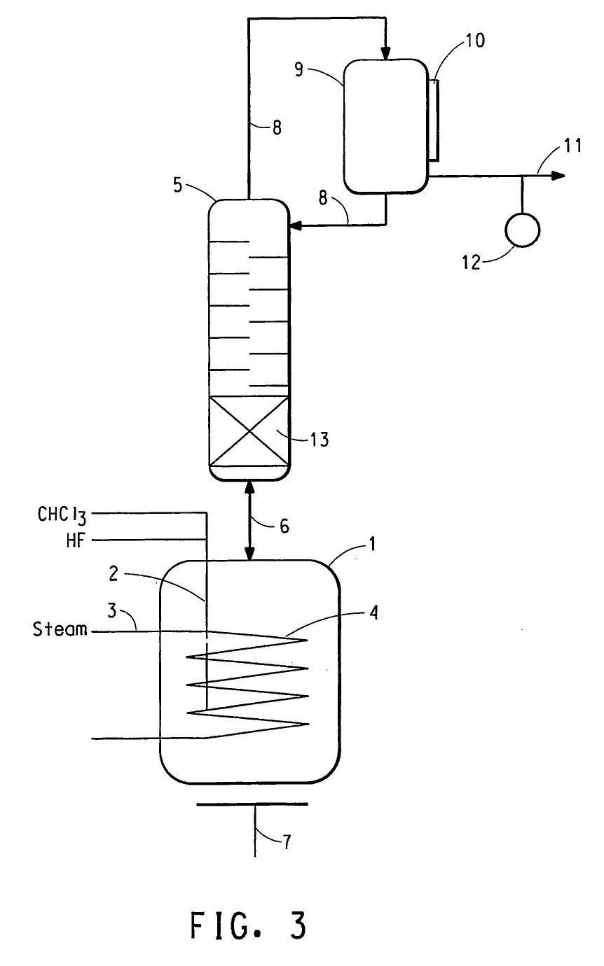

Practice of the process of this invention is further illustrated by reference to FIG. 3, which represents one possible embodiment of a reaction apparatus that can be adapted for operation in accordance with the present invention. A charge of liquid phase SbCl₅ is added to reactor 1. Liquid phase CHCl₃ and HF are added to reactor 1 via conduit 2. Heat is supplied to the reactor liquid phase by passing steam from conduit 3 through coil 4. The lower end of reflux column 5 is in fluid communication with reactor 1 via conduit 6. Reactor 1 rests on a pressure transducer 7 which allows for continuous monitoring of reactor 1 mass. Reactor vapor effluent comprising CHCl₃, CHCl₂F, CHClF₂, CHF₃, HCl and HF passes from reactor 1 upwardly through reflux column 5, resulting in reflux column vapor effluent comprising CHClF₂ and HCl. The upper end of reflux column 5 is in fluid communication via conduits 8 with condenser 9 containing a cooling jacket 10. Reflux column vapor effluent passes from the reflux column upper end to condenser 9. Brine cooling medium is circulated through cooling jacket 10 of condenser 9, thereby controlling the reflux ratio of condenser 9 so that a portion of the reflux column vapor effluent condenses and forms a condenser liquid effluent comprising CHClF₂, and a condenser vapor effluent comprising CHClF₂ and HCl. The condenser liquid effluent is passed from the condenser 9 to the upper end of reflux column 5, where it passes downwardly along the interior of the reflux column 5 and equilibrates with vapors therein, and at least a portion of which can pass to reactor 1 to join the reactor liquid phase. The condenser vapor effluent comprising CHClF₂ and HCl is removed from the condenser through conduit 11, and can thereafter be subjected to further processing, for example distillation and/or treatment with caustic, to remove HCl and form CHClF₂ product.

### Comparative Example 1

Reaction apparatus as described above and generally configured as in FIG. 3 is used. The reflux column comprises a cylinder containing a single packed bed of stainless steel #25 IMTP dumped packing material (from Norton Chemical Process Products Co., Ohio, USA). Steam flow to the heating coil 4 is controlled to maintain the average temperature of the reactor liquid phase at a setpoint between 70° and 90° C. The average temperature of the reactor liquid phase is the calculated mean of temperature measured at four different levels in the reactor liquid phase. The catalyst concentration is maintained at between 45 and 55 weight percent of the reactor liquid phase. The reaction apparatus pressure, measured at the reflux column upper end, is controlled between 195 and 220 psig. The temperature measured at a point halfway between the lower and upper ends of the reflux column (i.e., not at a point within the lower third of the theoretical stages of the reflux column) is controlled at between 30° and 40° C. by adjusting the flow of brine to the condenser.

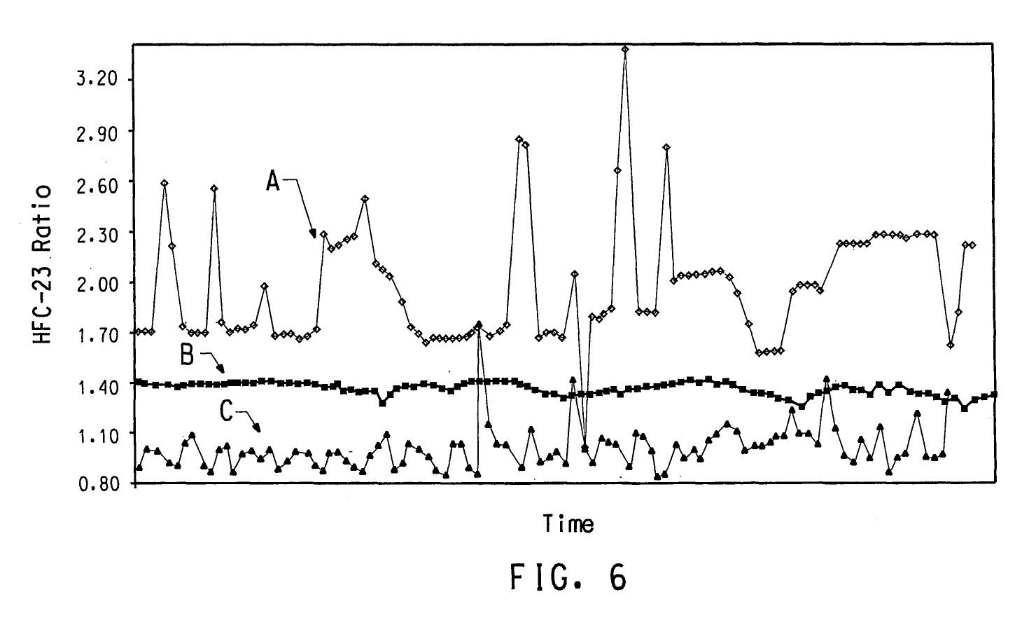

The CHF₃ ratio is measured every 2 hours over about 200 hours of steady state operation and is plotted versus time in FIG. 6 as line A. The maximum CHF₃ ratio measured over this time period is 3.35, the minimum CHF₃ ratio measured over this timeperiod is 1.01, and the average CHF₃ ratio measured over this timeperiod is 1.87.

### Example 1

The reaction apparatus and procedure of Comparative Example 1 is used in this example, with the following changes. The reactor liquid phase is maintained at a maximum mass that does not result in entrainment and/or flooding of the reactor liquid phase into reflux column 5. Brine cooling medium is circulated through cooling jacket 10 of condenser 9, and is used to set the reflux ratio of condenser 9 substantially constant during reactor steady state operation so that a portion of the reflux column vapor effluent condenses and forms a condenser liquid effluent comprising CHClF₂, and a condenser vapor effluent comprising CHClF₂ and HCl with a reduced amount of, or substantially free of, CHCl₂F and CHF₃. The reflux ratio averages 1.60 (minimum 1.41, maximum 1.79). The temperature at a point within the lower third of the theoretical stages of the reflux column averages 41.5° C. (minimum 37.9° C., maximum 43.4° C.). The amount of pentavalent antimony catalyst present in the reactor liquid phase averages 29.2 weight % (minimum 28.9 weight %, maximum 29.5 weight %). The CHCl₃/HF feed ratio to the reactor via conduit 2 averages 2.78 (minimum 2.48, maximum 3.01). The reaction apparatus pressure measured at the reflux column upper end averages 194.1 psig (minimum 193.1 psig, maximum 197.1 psig). The concentration value for each of CHCl₂F and CHF₃ in the condenser vapor effluent is measured by on-line gas chromatograph 12.

When the concentration of CHCl₂F in the condenser vapor effluent is measured to be higher than the CHCl₂F specification of 0.02 weight percent of CHCl₂F in the condenser vapor effluent (based on the combined weight of CHCl₂F and CHClF₂ in the condenser vapor effluent), and the concentration of CHF₃ is within the CHF₃ specification of 0.6 weight percent CHF₃ (based on the combined weight of CHF₃ and CHClF₂ in the condenser vapor effluent), the concentration of CHCl₂F is reduced to within the CHCl₂F specification by reducing the temperature at a point 13 within the lower third of the theoretical stages in the reflux column. The temperature at point 13 is reduced by reducing the heat input to the reactor liquid phase by reducing the amount of steam passing from conduit 3 through coil 4. The temperature at point 13 is reduced, and thereby the CHCl₂F concentration, as long as the CHF₃ concentration remains within specification. If the temperature reduction at point 13 causes the reactor liquid phase temperature to reach 68° C., then such temperature reduction is halted, and the reactor pressure increased by reducing the flow of condenser vapor effluent from the condenser through conduit 11.

When the concentration of CHF₃ in the condenser vapor effluent is measured to be higher than the aforementioned CHF₃ specification and the concentration of CHCl₂F is determined to be lower than the aforementioned CHCl₂F specification, the concentration of CHF₃ is reduced to within the CHF₃ specification by decreasing the reactor apparatus pressure by increasing the rate of removal of condenser vapor effluent from the condenser through conduit 11. This pressure is reduced, and thereby the CHF₃ concentration, as long as the CHCl₂F concentration does not rise and exceed the CHCl₂F specification. If such pressure reduction causes the reactor apparatus pressure to reach the minimum pressure at which the reactor apparatus can be operated before the amount of CHF₃ in the condenser vapor effluent is within the aforementioned CHF₃ specification, then the temperature at a point 13 within the lower third of the theoretical stages in the reflux column is increased by increasing the heat input to the reactor liquid phase by increasing the amount of steam passing from conduit 3 through coil 4.

The CHF₃ ratio is measured every 2 hours over about 200 hours of steady state operation and is plotted versus time in FIG. 6 as line B. The maximum CHF₃ ratio measured over this time period is 1.42, the minimum CHF₃ ratio measured over this time period is 1.15, and the average CHF₃ ratio measured over this time period is 1.33.

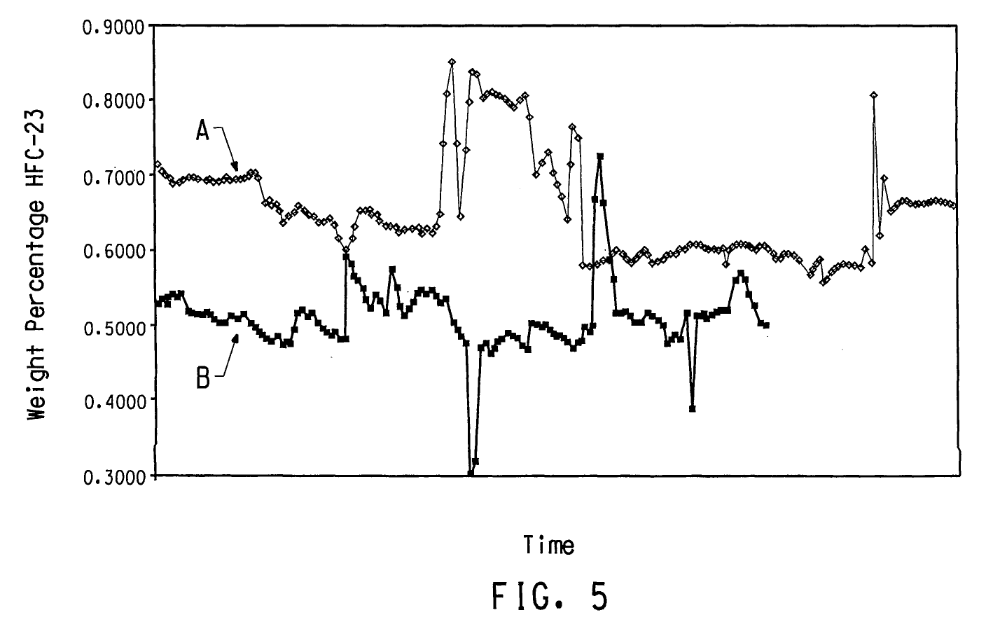

The CHF₃ concentration is measured every 2 hours over about 200 hours of steady state operation and is plotted versus time in FIG. 5 as line A. The maximum CHF₃ concentration measured over this time period is 0.85, the minimum CHF₃ concentration measured over this time period is 0.55, and the average CHF₃ concentration measured over this time period is 0.65.

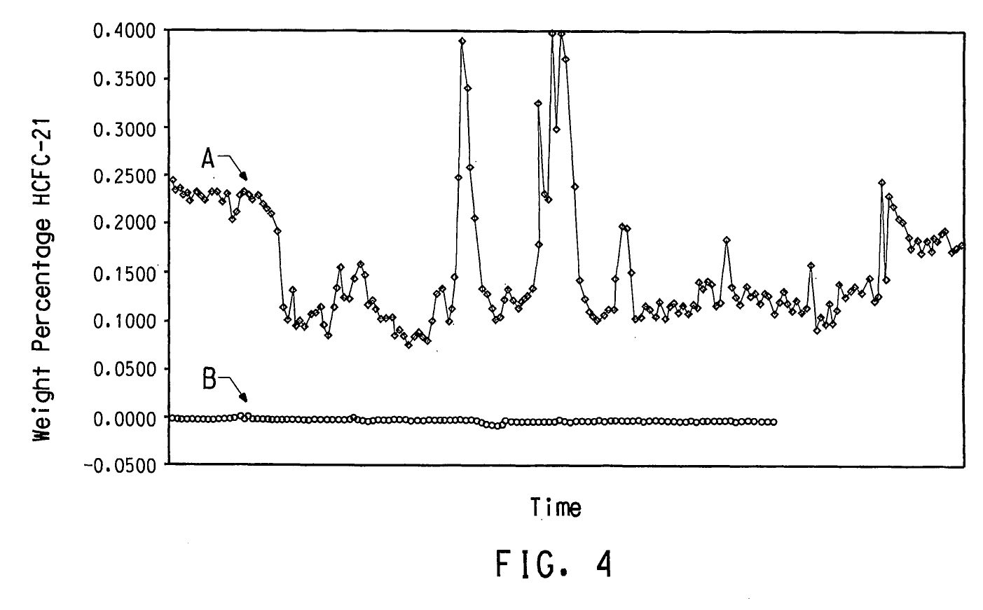

The CHCl₂F concentration is measured every 2 hours over about 200 hours of steady state operation and is plotted versus time in FIG. 4 as line A. The maximum CHCl₂F concentration measured over this time period is 0.4, the minimum CHCl₂F concentration measured over this time period is 0.08, and the average CHCl₂F concentration measured over this time period is 0.16.

### Example 2

The reaction apparatus and procedure of Example 1 is used in this example, with the following changes. The reflux column of Example 1 was replaced with a cylindrical column of identical dimensions, however, the bottom third of the length of the reflux column contained a packed bed of stainless steel #25 IMTP dumped packing material (from Norton Chemical Process Products Co., Ohio, USA) and the top two thirds of the reflux column contained 18 trays and no packing. The reflux ratio averages 1.59 (minimum 1.53, maximum 1.99). The temperature at a point within the lower third of the theoretical stages of the reflux column averages 45.9° C. (minimum 36.5° C., maximum 48.7° C.). The amount of pentavalent antimony catalyst present in the reactor liquid phase averages 28.4 weight % (minimum 27.6 weight %, maximum 29.2 weight %). The CHCl₃/HF feed ratio to the reactor via conduit 2 averages 2.78 (minimum 1.71, maximum 2.95). The reaction apparatus pressure measured at the reflux column upper end averages 199.8 psig (minimum 197.0 psig, maximum 201.4 psig).

The CHF₃ ratio is measured every hour over about 96 hours steady state operation and is plotted versus time in FIG. 6 as line C. The maximum CHF₃ ratio measured over this time period is 1.76, the minimum CHF₃ ratio measured over this time period is 0.8, and the average CHF₃ ratio measured over this time period is 1.03.

The CHF₃ concentration is measured every hour over about 96 hours steady state operation and is plotted versus time in FIG. 5 as line B. The maximum CHF₃ concentration measured over this time period is 0.73, the minimum CHF₃ concentration measured over this time period is 0.30, and the average CHF₃ concentration measured over this time period is 0.51.

The CHCl₂F concentration is measured every hour over about 96 hours steady state operation and is plotted versus time in FIG. 4 as line B. There is no CHCl₂F measured in the condenser vapor effluent over this time period.

1. (canceled)

2. The process of claim 25 wherein the reflux ratio of the condenser is maintained at a substantially constant value by controlling the cooling rate of the condenser; and wherein the mass of the reactor liquid phase is maintained by controlling the CHCl₃ feed flowrate to the reactor.

3. The process of claim 2 wherein the CHClF₂ production rate is controlled by controlling the HF feed flowrate to the reactor.

4. The process of claim 25 comprising measuring the concentration of each of CHCl₂F and CHF₃ in the condenser vapor effluent, and controlling the concentrations of CHCl₂F and CHF₃ in the condenser vapor effluent as necessary to provide a condenser vapor effluent wherein the concentration of each is below a designated specification limit set for it.

5. The process of claim 4 wherein the concentration of CHCl₂F in the condenser vapor effluent is greater than a set specification limit, and the concentration of CHCl₂F in the condenser vapor effluent is reduced by reducing the temperature at a point within the lower third of the theoretical stages of the reflux column by reducing the heat input to the reactor liquid phase.

6. The process of claim 4 wherein the concentration of CHF₃ in the condenser vapor effluent is greater than a set specification limit, and the concentration of CHF₃ in the condenser vapor effluent is reduced by reducing the pressure in the reactor, the reflux column and the condenser by increasing the rate at which the condenser vapor effluent is removed from the condenser.

7. The process of claim 25 wherein the concentration of CHCl₂F in the condenser vapor effluent is reduced by reducing the temperature at a point within the lower third of the theoretical stages of the reflux column by reducing the heat input to the reactor liquid phase.

8. The process of claim 25 wherein the concentration of CHF₃ in the condenser vapor effluent is reduced by reducing the pressure in the reactor, the reflux column and the condenser by increasing the rate at which the condenser vapor effluent is removed from the condenser.

9. The process of claim 25, wherein the reactor liquid phase temperature is maintained within the range of from about 68° C. to about 95° C.

10. The process of claim 9, wherein the reactor liquid phase temperature is maintained at a temperature of at least about 70° C.

11. (canceled)

12. (canceled)

13. The process of claim 25 wherein the pentavalent antimony catalyst is from about 25 weight percent to about 30 weight percent of said reactor liquid phase.

14. The process of claim 25 wherein the rate at which HF is added to said reactor during said contacting is less than about 0.5 pounds HF per hour, per pound of catalyst.

15. The process of claim 25 wherein the temperature at a point within the lower third of the theoretical stages in the reflux column is controlled to be from about 30° C. to about 60° C.

16. The process of claim 25 wherein the pressure measured at the reflux column upper end is from about 1,411 kPa (190 psig) to about 1,687 kPa (230 psig).

17. The process of claim 25 wherein the reflux ratio of the condenser is from about 1.0 to about 2.5.

18. The process of claim 17 wherein the reflux ratio of the condenser is from about 1.2 to about 2.0.

19. The process of claim 25 wherein the weight ratio of CHCl₃ to HF added to the reactor during (a) is from about 2.4 to about 3.2.

20. The process of claim 25 wherein the concentrations of CHCl₂F and CHF₃ in the condenser vapor effluent are controlled so that the condenser vapor effluent contains less than about 0.75 weight percent CHF₃, based on the combined weight of CHF₃ and CHClF₂ in the condenser vapor effluent, and less than about 0.1 weight percent CHCl₂F, based on the combined weight of CHCl₂F and CHClF₂ in the condenser vapor effluent.

21. The process of claim 20 wherein the concentration of CHF₃ in the condenser vapor effluent is controlled so that the condenser vapor effluent contains less than about 0.65 weight percent CHF₃, based on the combined weight of CHF₃ and CHClF₂ in said condenser vapor effluent.

22. The process of claim 20 wherein the concentration of CHCl₂F in the condenser vapor effluent is controlled so that the condenser vapor effluent contains less than about 0.05 weight percent of CHCl₂F, based on the combined weight of CHCl₂F and CHClF₂ in said condenser vapor effluent.

23. The process of claim 22 wherein the concentration of CHCl₂F in the condenser vapor effluent is controlled so that the condenser vapor effluent contains less than about 0.02 weight percent of CHCl₂F, based on the combined weight of CHCl₂F and CHClF₂ in said condenser vapor effluent.

24. (canceled)

25. A process for the manufacture of CHClF₂, comprising:
(a) contacting CHCl₃, HF and catalyst comprising pentavalent antimony in the liquid phase in a reactor to form a reactor liquid phase and a reactor vapor effluent comprising CHCl₃, CHCl₂F, HF, CHClF₂, CHF₃ and HCl, the pentavalent antimony catalyst being from about 25 weight percent to about 35 weight percent of said reactor liquid phase;
(b) passing the reactor vapor effluent to a reflux column having a lower and upper end, a lower portion that is packed and an upper portion that is trayed, the reflux column lower end in fluid communication with the reactor, to produce a reflux column vapor effluent comprising CHClF₂ and HCl;
(c) passing the reflux column vapor effluent from the reflux column upper end to a condenser in fluid communication with the reflux column upper end to produce a condenser liquid effluent comprising CHClF₂ and a condenser vapor effluent comprising CHClF₂ and HCl;
(d) controlling the concentration of CHCl₂F and CHF₃ in the condenser vapor effluent by:
(i) controlling the temperature at a point within the lower third of the theoretical stages of the reflux column by controlling the heat input to the reactor liquid phase;
(ii) controlling the pressure in the reactor, the reflux column and the condenser by controlling the rate at which the condenser vapor effluent is removed from the condenser;
(iii) maintaining the reflux ratio of the condenser at a substantially constant value; and
(iv) maintaining the reactor liquid phase at substantially the maximum mass that does not result in entrainment or flooding of the reflux column;
(e) passing the condenser liquid effluent to the reflux column upper end; and
(f) recovering CHClF₂ from said condenser vapor effluent.

26. The process of claim 25 wherein the reflux ratio of the condenser is maintained at a substantially constant value by controlling the cooling rate of the condenser; wherein the mass of the reactor liquid phase is maintained by controlling the CHCl₃ feed flowrate to the reactor; wherein the CHClF₂ production rate is controlled by controlling the HF feed flowrate to the reactor; and wherein the pentavalent antimony catalyst is from about 25 weight percent to about 30 weight percent of said reactor liquid phase.

27. CHClF₂ which is a product of the process of claim 25.

28. A refrigerant comprising CHClF₂ manufactured by the process of claim 25.

29. A method of producing a refrigerant, comprising
(1) manufacturing CHClF₂ in accordance with the process of claim 25; and
(2) mixing CHClF₂ manufactured in (1) with at least one compound selected from the group consisting of carbon dioxide, ammonia, hydrocarbons, hydrofluorocarbons, hydrochlorofluorocarbons, perfluorocarbons, hydrofluoroethers and perfluoroethers.

30. A polymer foam blowing blend comprising CHClF₂ manufactured by the process of claim 25.

31. A method of producing a polymer foam blowing blend, comprising
(1) manufacturing CHClF₂ in accordance with the process of claim 25; and
(2) blending CHClF₂ manufactured in (1) with at least one compound selected from the group consisting of dimethyl ether, carbon dioxide, hydrocarbons, hydrofluorocarbons, hydrochlorofluorocarbons, perfluorocarbons, hydrofluoroethers and perfluoroethers.

32. A compound of the formula CF₂═CFX, wherein X is selected from the group consisting of F and CF₃, produced by pyrolyzing CHClF₂ manufactured in accordance with the process of claim 25.

33. A method for producing at least one compound of the formula CF₂═CFX, wherein X is selected from the group consisting of F and CF₃, comprising
(1) manufacturing CHClF₂ in accordance with the process of claim 25; and
(2) pyrolyzing CHClF₂ manufactured in (1).

34. A method of producing a fluoropolymer, comprising
(1) manufacturing CHClF₂ in accordance with the process of claim 25;
(2) pyrolyzing CHClF₂ manufactured in (1) to produce at least one compound of the formula CF₂═CFX, wherein X is selected from the group consisting of F and CF₃; and
(3) polymerizing at least one compound of the formula CF₂═CFX produced in (2), optionally together with at least one comonomer.

35. A fluoropolymer produced by the method of claim 34.
